(19) United States
(12) Plant Patent Application Publication
(10) Pub. No.: US 2003/0204887 P1
(43) Pub. Date: Oct. 30, 2003

(54) Lonicera periclymenum plant named 'Scentsation'

(76) Inventors: John Edmonds, Hereford (GB); Elisabeth Wilms, Hereford (GB)

Correspondence Address: CHRISTIE, PARKER & HALE, LLP, P.O. BOX 7068, PASADENA, CA, 91109-7068, US

(21) Appl. No.: 10/425,898

(22) Filed: Apr. 28, 2003

(30) Foreign Application Priority Data
Apr. 29, 2002 (EP) 2002/0559

(51) Int. Cl. A01H 5/00

(52) U.S. Cl. PLT/226

(57) ABSTRACT

A new plant variety of Lonicera periclymenum characterized by its fragrant and abundant flowers.

### BACKGROUND OF THE INVENTION

The present invention relates to a new and distinct variety of Lonicera periclymenum L., which was discovered as a branch sport of an unnamed, unpatented variety of Lonicera periclymenum plant by John Edmunds and Elisabeth Wilms in Herefordshire, Great Britain. The varietal denomination of the new variety is ‘Scentsation’.

The genus Lonicera is included in the family Caprifoliaceae which comprises about 15 genera of shrubs growing in the northern and southern hemispheres. Lonicera comprises over 200 species of deciduous or evergreen shrubs, and erect or climbing shrubs, many of which possess desirable ornamental characteristics.

Lonicera periclymenum is a twining shrub native to Europe, North Africa and western Asia, with ovate to ovate-oblong leaves the undersides of which are colored bluish green. Flowers of L. periclymenum appear in whorls in summer and are two-lipped and pale yellow in color.

### SUMMARY OF THE INVENTION

The new variety was discovered as a branch sport of an unnamed, unpatented variety of Lonicera periclymenum and differs from its parent in having more fragrant and more abundant flowers. Asexual reproduction of the new variety by stem cuttings, performed in Herefordshire, Great Britain and Vacaville, Calif. have confirmed that the distinctive characteristics of the new variety are stable and transmitted to succeeding generations, and the new variety reproduces true to type.

### COMPARISON WITH PARENT

‘Scentsation’ is distinguished from its parent and all other varieties of Lonicera periclymenum of which I am aware in having more fragrant and more abundant flowers.

### BRIEF DESCRIPTION OF ILLUSTRATIONS

The accompanying photographic illustrations show a specimen of the new cultivar as true to color as is reasonably possible to make in an illustration of this character.

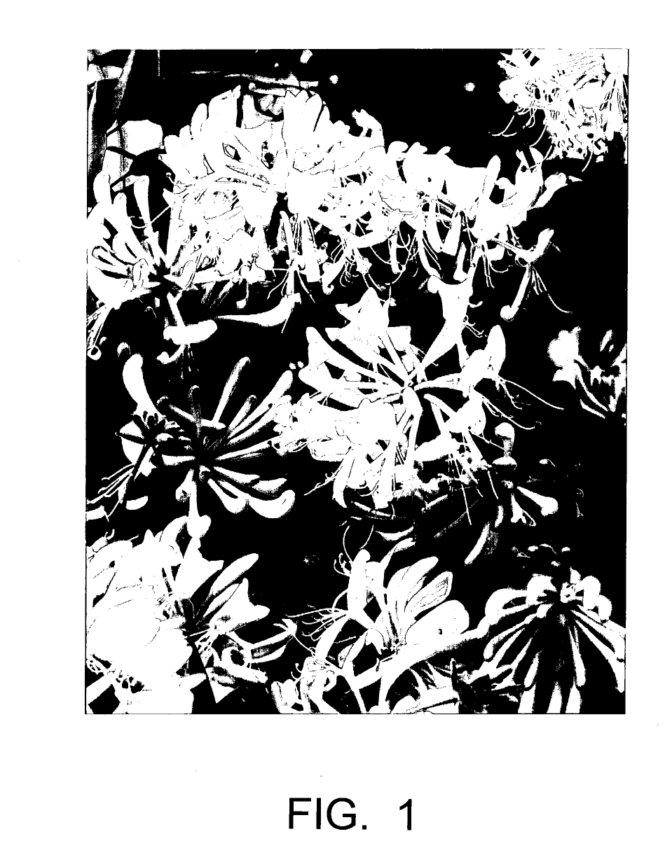

FIG. 1 illustrates the typical flowers of ‘Scentsation’.

### DETAILED DESCRIPTION OF THE NEW VARIETY

‘Scentsation’ has not been observed under all possible environmental, cultural and light conditions. The following observations and descriptions are of one-year-old plants grown in 1-gallon containers in Vacaville, Calif. In this description, color references are to The Royal Horticultural Society Colour Chart (1995) and terminology used in the color descriptions herein refers to plate numbers in this color chart. Phenotypic expression may vary with light intensity, cultural and environmental conditions.

Classification:

Botanical.—Lonicera periclymenum L. ‘Scentsation’.

Parentage.—Branch sport of unnamed, unpatented variety of Lonicera periclymenum.

Propagation.—By vegetative cuttings.

Plant:

Size.—Mature plant can climb and spread to about 10 meters at maturity.

Habit.—A scrambling, twining vine that can be grown on the ground, or allowed to climb screens or trellises.

Branching.—Opposite, sparse.

Leaf description.—Arrangement: Simple, opposite on stems Leaf shape: Ovate to sublanceolate Apex: Acute to obtuse Base: Cuneate Leaf size: From 30 to 60 mm long and 15 to 28 mm wide Leaf margin: Entire Leaf texture and substance Adaxial: Smooth, glossy; slightly impressed veins Abaxial: Pubescent Immature leaf color Adaxial: Near Green Group 137B Abaxial: Near Green Group 138B Midrib: Near Yellow-Green Group 145B Mature leaf color Adaxial: Near Green Group 137C Abaxial: Near Greyed-Green Group 191B Mature Stem color: Near Green Group 143A.

Petioles.—Young Leaves: Subsessile Mature Leaves Size: From 1 to 1.5 mm long Color: Near Greyed-Orange Group 165B.

Hardiness.—USDA Zone 5 (−20° F. to −10° F.).

Vigor.—Moderate, 1 to 2 m per year.

Pests/Diseases.—No pest or disease problems noted.

Inflorescence:

Bloom period.—March through May.

Lastingness of individual bloom.—2 weeks.

Shape of inflorescence.—Domed umbel.

Flower arrangement.—Umbel in whorls.

Flower form.—Tubular, two-lipped.

Flower size.—Length: 30 to 38 mm Width at Base: Approximately 2 mm.

Color.—New Flowers: Near Yellow Group 4D Older Flowers: Near Yellow Group 8A-B.

Buds.—Shape: Tubular, inflated, falcate Size: Up to 35 mm lone, 8 mm wide.

Peduncles.—5 mm.

Pedestal.—Sessile.

Fragrance.—Sweet, moderately strong.

Reproductive Organs:

Stamens.—Number: 5 Length: 25 to 30 mm long Color: Near Yellow Group 8A-B.

Pistils.—1.

Stigma.—1, Near Yellow Green Group 144B.

Anther color.—Near Yellow-Orange Group 16B.

Fruit.—None observed.

I claim:

1. A new plant variety of Lonicera periclymenum of the variety substantially as shown and described.
